(19) United States
(12) Patent Application Publication
(10) Pub. No.: US 2022/0151982 A1
(43) Pub. Date: May 19, 2022

(54) CANNABINOID COMPOSITIONS AND USE THEREOF

(71) Applicant: SCICANN THERAPEUTICS INC., Toronto (CA)

(72) Inventor: Zohar KOREN, Gan Ner (IL)

(21) Appl. No.: 17/598,883

(22) PCT Filed: Mar. 26, 2020
(86) PCT No.: PCT/IB2020/052875

§ 371 Date: Sep. 28, 2021

Related U.S. Application Data
(60) Provisional application No. 62/825,095, filed on Mar. 28, 2019.

(51) Int. Cl. A61K 31/352; A61K 31/05; A61K 31/337; A61K 31/475; A61K 31/513; A61K 31/704; A61K 38/14; A61K 33/243; A61K 45/06; A61K 31/165; A61K 31/65; A61K 31/496; A61P 31/04; A61P 35/00

(52) U.S. Cl. CPC A61K 31/352; A61K 31/05; A61K 31/337; A61K 31/475; A61K 31/513; A61K 31/704; A61P 35/00; A61K 33/243; A61K 45/06; A61K 31/165; A61K 31/65; A61K 31/496; A61P 31/04; A61K 38/14

(57) ABSTRACT

Methods of rapidly inhibiting efflux from a cell, sensitizing a drug-resistant cell to a drug and treating a subject with a drug-resistant pathology, by administering tetrahydrocannabinolic acid (THCa), cannabidiol (CBD) or a combination thereof are provided. Pharmaceutical compositions comprising THCa, CBD or a combination thereof and a drug are also provided.

### CROSS-REFERENCE TO RELATED APPLICATIONS

This application claims the benefit of priority of U.S. Patent Application No. 62/825,095 titled “CANNABINOID COMPOSITIONS AND USE THEREOF”, filed Mar. 28, 2019, the contents of which is incorporated herein by reference in its entirety.

### FIELD OF INVENTION

The present invention is in the field of cannabinoid therapeutics.

### BACKGROUND OF THE INVENTION

Drug-resistant cancers and antibiotic resistant bacteria are two of the most daunting problems facing the health care industry. While new drugs are constantly being sought, the rate at which successful new entities are found is very low. A treatment that can sensitize a cell to a drug and convert a resistant pathogen into a treatable one is therefore greatly needed.

### SUMMARY OF THE INVENTION

The present invention provides methods of rapidly inhibiting efflux from a cell, sensitizing a drug-resistant cell to a drug and treating a subject with a drug-resistant pathology, by administering tetrahydrocannabinolic acid (THCa), cannabidiol (CBD) or a combination thereof. Pharmaceutical composition comprising THCa, CBD or a combination thereof and a drug are also provided.

According to another aspect, there is provided a pharmaceutical composition comprising tetrahydrocannabinolic acid (THCa), and a drug selected from an anti-cancer drug, and an antibiotic drug.

According to another aspect, there is provided a pharmaceutical composition comprising cannabidiol (CBD), and an antibiotic drug.

According to another aspect, there is provided a pharmaceutical composition comprising THCa, CBD or a combination thereof, a drug selected from an anti-cancer drug, and an antibiotic drug and a pharmaceutically acceptable carrier, excipient or adjuvant.

According to some embodiments, the anti-cancer drug, the antibiotic or both are not an antiepileptic, nor an anti-inflammatory.

According to some embodiments, the THCa, CBD or a combination thereof and the drug are in a single dosage form.

According to some embodiments, the THCa, CBD or a combination thereof is enriched in the pharmaceutical composition.

According to some embodiments, the anti-cancer drug is a chemotherapeutic, optionally wherein the chemotherapeutic is selected from doxorubicin, paclitaxel, Cisplatin and 5-FU.

According to some embodiments, the antibiotic drug is selected from tetracycline, gentamycin, chloramphenicol, ciprofloxacin, rifampicin, and vancomycin.

According to some embodiments, the pharmaceutical composition of the invention is for use in at least one of:
a. inhibiting efflux from a cell in a subject;
b. sensitizing a drug-resistant cell in a subject to the drug; and
c. treating a drug-resistant condition or disease in a subject in need thereof by coadministration with the drug.

According to another aspect, there is provided a method of inhibiting efflux from a cell through a membrane channel, comprising contacting the cell with tetrahydrocannabinolic acid (THCa), thereby inhibiting efflux from a cell.

According to another aspect, there is provided a method of inhibiting efflux from a cell through a membrane channel, comprising contacting the cell with tetrahydrocannabinolic acid (THCa), cannabidiol (CBD) or a combination thereof, thereby inhibiting efflux from a cell.

According to some embodiments, the cell is a chemotherapy resistant cell.

According to some embodiments, the chemotherapy resistant cell is a multi-drug-resistant (MDR) cell.

According to some embodiments, the cell is a cancer cell, and wherein the cancer is selected from ovarian cancer, pancreatic cancer, and lung cancer.

According to some embodiments, the cell is an antibiotic resistant cell.

According to some embodiments, the antibiotic resistant cell is a Methicillin-resistant Staphylococcus aureus (MRSA) cell.

According to another aspect, there is provided a method of sensitizing a drug-resistant cell to the drug, the method comprising contacting the cell with THCa, thereby sensitizing a drug-resistant cell to the drug.

According to another aspect, there is provided a method of sensitizing a drug-resistant bacterium to the drug, the method comprising contacting the cell with CBD, thereby sensitizing a drug-resistant bacterium to the drug.

According to another aspect, there is provided a method of sensitizing a drug-resistant cell to the drug, the method comprising contacting the cell with THCa, CBD or a combination thereof, thereby sensitizing a drug-resistant cell to the drug.

According to some embodiments, the drug-resistant cell comprises efflux-mediated drug-resistance.

According to some embodiments, the drug is selected from an anti-cancer drug, an antibiotic, an antipsychotic, an anti-androgen, an immunosuppressant, a lipid lowering drug, an antihistamine, a steroid, a dopamine antagonist, a protein inhibitor, a cardiac drug, an antiemetic, an antidiarrheal, and antigout and an anti-fungal.

According to some embodiments, the anti-cancer drug is selected from a chemotherapeutic, an anthracycline, a vinca alkaloid, a taxane, a podophyllotoxin derivative, a PARP inhibitor, a folate based anti-metabolite, an alkylating agent, an epothilone, a histone deacetylase inhibitor, a topoisomerase I or II inhibitor, a kinase inhibitor, a nucleotide analog or precursor analog, a podophyllotoxin derivative, a platinum based agent, or a retinoid.

According to some embodiments, the anticancer drug is a chemotherapeutic selected from doxorubicin, paclitaxel, cisplatin and 5-FU.

According to some embodiments, the antibiotic drug is selected from tetracycline, gentamycin, chloramphenicol, ciprofloxacin, rifampicin, and vancomycin.

According to some embodiments, the drug-resistant cell is a cancer cell in a subject, and the method further comprises administering the drug to the subject, thereby treating cancer in the subject.

According to some embodiments, the drug-resistant cancer is a multidrug-resistant cancer.

According to some embodiments, the cancer is selected from ovarian cancer, pancreatic cancer, and lung cancer.

According to some embodiments, the drug-resistant cell is a pathogen in a subject, and the method further comprises administering the drug to the subject, thereby treating the an infection by the pathogen in the subject.

According to some embodiments, the pathogen is an antibiotic resistant bacterium.

According to some embodiments, the antibiotic resistant bacterium is MRSA.

According to some embodiments, the administering is concomitant with the contacting or subsequent to the contacting.

According to another aspect, there is provided a kit comprising:
a. THCa,
b. a drug selected from an anti-cancer drug, and an antibiotic drug; and
c. a label stating the THCa is for use in combination with the drug selected from an anti-cancer drug, and an antibiotic drug.

According to another aspect, there is provided a kit comprising CBD, an antibiotic drug and a label stating the CBD is for use in combination with the antibiotic drug.

According to another aspect, there is provided a kit comprising THCa, CBD or both; and at least one of
a. a drug selected from an anti-cancer drug, and an antibiotic drug; and
b. a label stating the THCa, CBD or both are for use in combination with the drug selected from an anti-cancer drug, and an antibiotic.

In some embodiments, the anti-cancer drug is a chemotherapeutic selected from doxorubicin, paclitaxel, Cisplatin and 5-FU.

In some embodiments, the antibiotic drug is selected from tetracycline, gentamycin, chloramphenicol, ciprofloxacin, rifampicin, and vancomycin.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: A graph of sensitization of resistant ovarian cancer cell line to Doxorubicin therapy, comparable to non-resistant cell line sensitivity levels, through combination of Dox with THCa.

FIG. 2: A graph of dose dependent potentiating effect of THCa on Doxorubicin cytotoxicity in resistant ovarian cancer cell line.

FIG. 3: A graph of the synergistic cytotoxic effect of THCa and Doxorubicin in resistant ovarian cancer cell line

FIG. 4: A graph of sensitization of resistant ovarian cancer cell line to Doxorubicin therapy, comparable to non-resistant cell line sensitivity levels, through combination of Dox with CBD.

FIG. 5: A graph of dose dependent potentiating effect of CBD on Doxorubicin cytotoxicity in resistant ovarian cancer cell line.

FIG. 6: A graph of the synergistic cytotoxic effect of low dose CBD and Doxorubicin in resistant ovarian cancer cell line.

FIG. 7: A graph of the synergistic cytotoxic effect of higher dose CBD and Doxorubicin in resistant ovarian cancer cell line.

FIGS. 8A-B: Line graphs of the amount of Doxorubicin efflux out of the cells and into the media in the presence and absence of (8A) THCa or (8B) CBD. Efflux is presented as a percentage of the highest efflux observed.

FIG. 9: A graph of the synergistic cytotoxic effect of THCa and Taxol in resistant ovarian cancer cell line.

FIG. 10: A graph of the synergistic cytotoxic effect of THCa and Cisplatin in resistant ovarian cancer cell line.

FIG. 11: A graph of the synergistic cytotoxic effect of THCa and 5-FU in resistant pancreatic cancer cell line.

FIG. 12: A graph of the synergistic cytotoxic effect of THCa and Vincristine in resistant lung cancer cell line.

FIG. 13: A picture of the chemical structure of CBD.

FIG. 14: A picture of the chemical structure of THCa.

### DETAILED DESCRIPTION OF THE INVENTION

The present invention, in some embodiments, provides methods of inhibiting efflux from a cell through an efflux pump membrane channel. The present invention further concerns a method of sensitizing a drug-resistant cell to the drug and treating a drug-resistant pathology. A pharmaceutical composition comprising THCa, CBD or a combination thereof and a drug is also provided.

### Efflux Inhibition

By a first aspect, there is provided a method of inhibiting efflux from a cell, the method comprising contacting the cell with a cannabinoid or derivative thereof, thereby inhibiting efflux from the cell.

As used herein, the term “efflux” refers to the transport of a particle out of a cell. In some embodiments, the efflux is via active transport of the particle out of the cell. In some embodiments, active transport is energy-dependent transport. In some embodiments, efflux is flowing out of the cell. In some embodiments, the efflux is through a membranal protein in the cell. In some embodiments, the membranal protein is a membrane channel. In some embodiments, the membrane channel is an efflux pump.

In some embodiments, the membrane channel is a prokaryotic membrane channel. In some embodiments, the membrane channel is a bacterial membrane channel. In some embodiments, the membrane channel is a eukaryotic membrane channel. In some embodiments, the membrane channel is a mammalian membrane channel. In some embodiments, the membrane channel is a human membrane channel. In some embodiments, a bacterial membrane channel is selected from a major facilitator superfamily channel, an ATP-binding cassette superfamily channel, a small multidrug-resistance family channel, a resistance-nodulation cell division superfamily membrane channel and a multi-antimicrobial extrusion protein family channel. In some embodiments, a human membrane channels is selected from monocarboxylate transporters, multiple drug-resistance proteins, peptide transporters and Na+ phosphate transporters. Membrane channel for efflux are well known in the art and include, but are not limited to, Multidrug Resistance-associated Protein 1 (MRP1), Multidrug Resistance Protein 1 (MDR1) and Breast Cancer Resistance Protein (BCRP), BCRP, ATP-binding cassette transporter ABCA1 (ABCA1), AcrAB, MtrCDE, and AcrAB. In some embodiments, the membrane channel is MRP1. In some embodiments, the ABCC1 gene codes for MRP1. In some embodiments, the membrane channel is MDR1. MDR1 is also known as P-glycoprotein 1. In some embodiments, the membrane channel is BCRP. In some embodiments, the ABCG2 gene encodes BCRP. In some embodiments, the membrane channel is ABCA1. ABCA1 is also known as Cholesterol Efflux Regulatory Protein (CERP). In some embodiments, the membrane channel is ABCA1.

In some embodiments, the cell is a target cell. In some embodiments, the cell is a prokaryotic cell. In some embodiments, the cell is a eukaryotic cell. In some embodiments, the cell is a bacterial cell. In some embodiments, the cell is a mammalian cell. In some embodiments, the cell is a human cell. In some embodiments, the cell is an antibiotic-resistant bacterial cell. In some embodiments, the antibiotic-resistant bacterial cell is a Methicillin-resistant Staphylococcus aureus (MRSA) cell. In some embodiments, the antibiotic is selected from tetracycline, gentamycin, chloramphenicol, ciprofloxacin, rifampicin, and vancomycin. In some embodiments, the antibiotic is tetracycline. In some embodiments, the antibiotic is gentamycin. In some embodiments, the antibiotic is chloramphenicol. In some embodiments, the antibiotic is ciprofloxacin. In some embodiments, the antibiotic is rifampicin. In some embodiments, the antibiotic is vancomycin. In some embodiments, the cell is a fungal cell. In some embodiments, the cell is multi-drug-resistant. In some embodiments, the cell is a neuron. In some embodiments, the cell is a pathogenic cell. In some embodiments, the cell is a disease cell.

In some embodiments, the cell is a cancer cell. In some embodiments, the cancer is a drug-resistant cancer. In some embodiments, the cancer is a chemotherapy resistant cancer. In some embodiments, the cancer is a multi-drug-resistant cancer. In some embodiments, the cancer is ovarian cancer. In some embodiments, the cancer is pancreatic cancer. In some embodiments, the cancer is lung cancer. In some embodiments, the lung cancer is small cell lung cancer. Examples of drug-resistant cancers include, but are not limited to lung cancer, pancreatic cancer, ovarian cancer, colorectal cancer, leukemia, lymphoma, breast cancer, melanoma and glioblastoma. In some embodiments, the cancer is a cancer treatable with doxorubicin. In some embodiments, the cancer is a cancer treatable with paclitaxel. In some embodiments, the cancer is a cancer treatable with cisplatin. In some embodiments, the cancer is a cancer treatable with fluorouracil (5-FU). In some embodiments, a cancer treatable with doxorubicin is selected from breast cancer, bladder cancer, Kaposi sarcoma, lymphoma, thyroid cancer, multiple myeloma and leukemia. In some embodiments, a cancer treatable with paclitaxel is selected from breast cancer, ovarian cancer, lung cancer, cervical cancer, Kaposi sarcoma, and pancreatic cancer. In some embodiments, a cancer treatable with cisplatin is selected from testicular cancer, carcinoma, germ cell tumors, ovarian cancer, cervical cancer, breast cancer, bladder cancer, head and neck cancer, lung cancer, esophageal cancer, mesothelioma, glioblastoma and brain cancer. In some embodiments, a cancer treatable with 5-FU is selected from breast cancer, bowel cancer, skin cancer, stomach cancer, esophageal cancer, colorectal cancer and pancreatic cancer. In some embodiments, the cancer is selected from ovarian cancer, pancreatic cancer, lung cancer, breast cancer, bladder cancer, Kaposi sarcoma, lymphoma, leukemia, cervical cancer, testicular cancer, head and neck cancer, esophageal cancer, mesothelioma, glioblastoma, brain cancer, bowel cancer, skin cancer, thyroid cancer, carcinoma, germ cell tumors, multiple myeloma, colorectal cancer and stomach cancer.

In some embodiments, the cell is an efflux membrane channel positive cell. In some embodiments, the cell comprises increased efflux membrane channel expression. In some embodiments, the increase is as compared to a healthy control cell. In some embodiments, the increase is at least a 10, 20, 30, 40, 50, 60, 70, 80, 90, 100, 150, 200 or 250% increase. Each possibility represents a separate embodiment of the invention. In some embodiments, the cell is an efflux positive cell. Routine methods of measuring efflux are well known in the art. Many such assays are also available commercially, such as dye efflux assays (Calcein assay and Hoeschst assay for example, Solvo Biotechnology) and MDR1 assays (Merck). Methods of testing efflux are also described hereinbelow.

In some embodiments, the cell is in vivo. In some embodiments, the cell is in a subject. In some embodiments, the cell is in vitro. In some embodiments, the cell is on a surface. In some embodiments, the cell is ex vivo.

In some embodiments, the inhibiting is rapid inhibition. In some embodiments, rapid is in 1, 2, 3, 4, 5, 6, 7, 8, 9, 10, 12, 18, or 24 hours, or less. Each possibility represents a separate embodiment of the invention. In some embodiments, rapid is in less than 1, 2, 3, 4, 5, 6, 7, 8, 9, 10, 12, 18, or 24 hours. Each possibility represents a separate embodiment of the invention. In some embodiments, rapid is in at most 1, 2, 3, 4, 5, 6, 7, 8, 9, 10, 12, 18, or 24 hours. Each possibility represents a separate embodiment of the invention. In some embodiments, the rapid inhibition occurs in not more than 4 hours.

As used herein, a “cannabinoid” refers to a molecule that binds to a cannabinoid receptor. In some embodiments, the cannabinoid is a phytocannabinoid. As used herein, a “phytocannabinoid” is a cannabinoid that originates in nature from the cannabis plant. In some embodiments, the cannabinoid is not psychoactive. Examples of cannabinoids include, but are not limited to tetrahydrocannabinol (THC), tetrahydrocannabinolic acid (THCa), tetrahydrocannabivarin (THCv), tetrahydrocannabivarinic acid (THCva), CBO, cannabinol (CBN), cannabinol propyl variant (CBNv), cannabicyclol (CBL), cannabigerol (CBG), cannabigerol propyl variant (CBGv), cannabidiol (CBD), cannabidiolic acid (CBDa), cannabidivarin (CBDv), cannabichromene (CBC), and cannabichromenic acid (CBCa). In some embodiments, a cannabinoid is contacted. In some embodiments, the cannabinoid is selected from CBD and THCa. In some embodiments, the cannabinoid is THCa. In some embodiments, the cannabinoid is CBD. In some embodiments, the cannabinoid is a combination of THCa and CBD. In some embodiments, the cannabinoid is selected from THCa, CBD and a combination thereof. In some embodiments, the cannabinoid is not THC.

In some embodiments, the cannabinoid is substantially devoid of THC. In some embodiments, the cannabinoid is not CBN. In some embodiments, THC is (−)-trans-Δ⁹-tetrahydrocannabinol. In some embodiments, the cannabinoid is a naturally occurring cannabinoid. In some embodiments, the cannabinoid is a synthetic cannabinoid. In some embodiments, the cannabinoid is an enriched cannabinoid. In some embodiments, the cannabinoid is a purified cannabinoid. In some embodiments, the cannabinoid is an isolated cannabinoid. In some embodiments, the cannabinoid is depleted of THC.

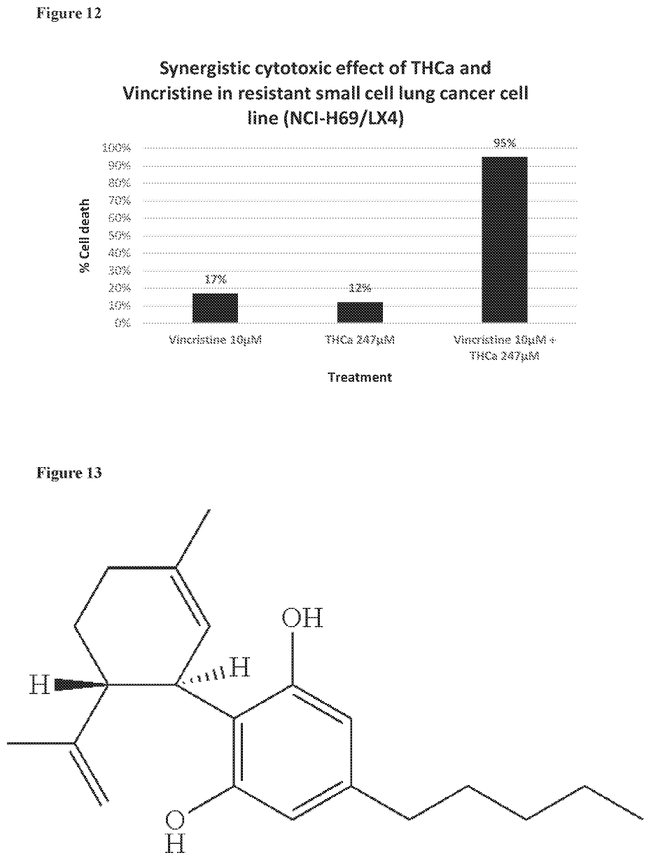

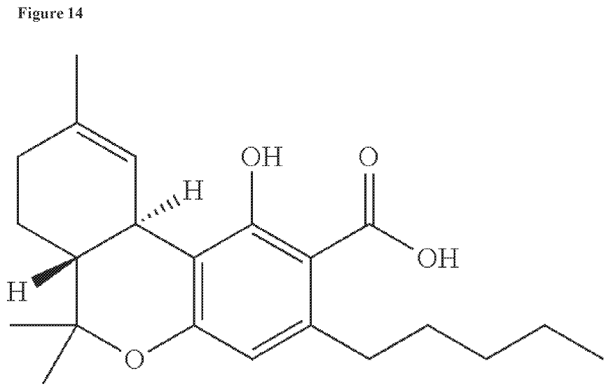

CBD is also termed 2-[(6R)-3-Methyl-6-prop-1-en-2-yl-lcyclohex-2-envyl]-5pentylbenzene-1,3-diol, and has the molecular formula C₂₁H₃₀O₂. The chemical structure of CBD is shown in FIG. 12. THCa, is also termed 2-COOH-THC, is a precursor of THC, is non-psychoactive, and has the molecular formula C₂₂H₃₀O₄. The chemical structure of THCa is shown in FIG. 13.

The term “derivative” as used herein, refers to a molecule generated from a cannabinoid. In some embodiments, the derivative is a derivative that has an efflux inhibiting effect. In some embodiments, the derivative is a derivative that has a sensitizing effect.

### Drug Sensitization

By another aspect, there is provided a method of sensitizing a drug-resistant cell to the drug, the method comprising contacting the cell with a cannabinoid or derivative thereof, thereby sensitizing a drug-resistant cell to the drug.

By another aspect, there is provided a method of killing a drug-resistant cell, the method comprising contacting the cell with a cannabinoid or derivative thereof and the drug, thereby killing a drug-resistant cell.

In some embodiments, the sensitizing is rapid sensitizing. In some embodiments, the sensitizing occurs in vitro. In some embodiments, the sensitizing occurs ex vivo. In some embodiments, the sensitizing occurs in vivo. In some embodiments, the sensitizing occurs in a subject. In some embodiments, the cell is in a subject. In some embodiments, the sensitizing is treating the subject. In some embodiments, the killing is treating the subject. In some embodiments, the cell is a target cell.

In some embodiments, the drug-resistant cell is a prokaryotic cell. In some embodiments, the drug-resistant cell is a eukaryotic cell. In some embodiments, the drug-resistant cell is a bacterial cell. In some embodiments, the drug-resistant cell is a mammalian cell. In some embodiments, the drug-resistant cell is a human cell. In some embodiments, the drug-resistant cell is an antibiotic-resistant bacterial cell. In some embodiments, the drug-resistant cell is a fungal cell. In some embodiments, the drug-resistant cell is multi-drug-resistant. In some embodiments, the drug-resistant cell is a neuron. In some embodiments, the drug-resistant cell is a pathogenic cell. In some embodiments, the drug-resistant cell is a disease cell. In some embodiments, the drug-resistant cell is a cancer cell. In some embodiments, the drug-resistance is efflux-mediated drug-resistance. In some embodiments, the drug-resistant cell is a target cell.

In some embodiments, the drug is an antibiotic. In some embodiments, the drug is an anti-fungal. In some embodiments, the drug is an anti-cancer drug. In some embodiments, the drug is an anti-androgen drug. In some embodiments, the drug is an antipsychotic. In some embodiments, the drug is an immunosuppressive drug. In some embodiments, the drug is a metabolic drug. In some embodiments, the drug is an antihistamine. In some embodiments, the drug is a steroid. In some embodiments, the drug is a protease inhibitor. In some embodiments, the drug is a cardiac drug. In some embodiments, the drug is an antiemetic. In some embodiments, the drug is an anti-diarrheal. In some embodiments, the drug is an antigout drug. In some embodiments, the drug is a neurological drug. In some embodiments, the drug is a dopamine antagonist. In some embodiments, the neurological drug is a dopamine antagonist. In some embodiments, the antipsychotic is a dopamine antagonist. In some embodiments, the anti-cancer drug is a chemotherapeutic. In some embodiments, the metabolic drug is a lipid-lowering drug. In some embodiments, the protease inhibitor is an HIV protease inhibitor. In some embodiments, the drug is a drug that can be effluxed from a cell by a membrane channel. In some embodiments, the drug is a drug that is actively transported out of a cell. Examples of such drugs include, but are not limited to chemotherapeutics, alkylating agents, anthracyclines, vinca alkaloids, taxanes, epothilones, histone deacetylase inhibitors, topoisomerase I and II inhibitors, kinase inhibitors, nucleotide analogs and precursor analogs, podophyllotoxin derivatives, PARP inhibitors, platinum-based agents, retinoids and folate based anti-metabolites.

In some embodiments, the chemotherapeutic is doxorubicin. In some embodiments, the chemotherapeutic is paclitaxel. In some embodiments, the chemotherapeutic is cisplatin. In some embodiments, the chemotherapeutic is 5-FU. In some embodiments, the anti-cancer drug is selected from: Doxorubicin, Daunorubicin, Vinblastine, Vincristine, Actinomycin D, Paclitaxel, Teniposide, Etoposide, Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Docetaxel, Doxifluridine, Epirubicin, Epothilone, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Pemetrexed, Tioguanine, Topotecan, Valrubicin, Vemurafenib and Vindesine. In some embodiments, the immunosuppressive drug is selected from Cyclosporin A and FK506. In some embodiments, the metabolic drug is Lovastatin. In some embodiments, the lipid-lowering drug is Lovastatin. In some embodiments, the antihistamine is Terfenadine. In some embodiments, the steroid is selected from: Aldosterone, Hydrocortisone, Cortisol, Corticosterone, and Dexamethasone. In some embodiments, the dopamine antagonist is Domperidone. In some embodiments, the HIV protease inhibitor is selected from: Amprenavir, Indinavir, Nelfinavir, Ritonavir, and Saquinavir. In some embodiments, the cardiac drug is selected from Digoxin and Quinidine. In some embodiments, the antiemetic drug is Ondansetron. In some embodiments, the antidiarrheal is loperamide. In some embodiments, the antigout drug is Colchicine.

In some embodiments, the antibiotic is selected from tetracycline, gentamycin, chloramphenicol, ciprofloxacin, rifampicin, and vancomycin. In some embodiments, the antibiotic is tetracycline. In some embodiments, the antibiotic is gentamycin. In some embodiments, the antibiotic is chloramphenicol. In some embodiments, the antibiotic is ciprofloxacin. In some embodiments, the antibiotic is rifampicin. In some embodiments, the antibiotic is vancomycin.

In some embodiments, efflux comprises efflux of a molecule. In some embodiments, the molecule is a drug. In some embodiments, the molecule is a dye. In some embodiments, the molecule is a synthetic molecule. In some embodiments, the molecule is a naturally occurring molecule. In some embodiments, the molecule is a cytotoxic molecule.

In some embodiments, sensitizing comprises an increase in the efficacy of the drug. In some embodiments, the increase is as compared to the effect of the drug when the cannabinoid is not contacted. In some embodiments, the increase is at least a 10, 20, 30, 40, 50, 60, 70, 80, 90, 100, 150, 200, 250, 300, 350, 400, 450, or 500% increase in efficacy. Efficacy can be measured by any means such as is routinely used to monitor the drugs effect. For example, the effect of a chemotherapeutic or antibiotic could be killing of the cell and so the percent of dead cells might be used to measure efficacy. Similarly, the time it takes the drug to have its effect could have been shortened, or the dose needed could have been decreased. The forgoing are merely examples and an increase in efficacy by any measure can be considered a sensitizing. In some embodiments, sensitizing is increasing cell death. In some embodiments, sensitizing turns a resistant cell to non-resistant. In some embodiments, sensitizing turns an ineffective drug into an effective one.

In some embodiments, the method further comprises contacting the cell with the drug. In some embodiments, the cell is in a subject and the method further comprises administering the drug to the subject. In some embodiments, the contacting the cannabinoid and contacting/administering the drug are concomitant. In some embodiments, the contacting the cannabinoid is prior to the administering/contacting with the drug. In some embodiments, contacting with the cannabinoid comprises contacting with a pharmaceutical composition comprising the cannabinoid. In some embodiments, contacting with the drug comprises contacting with a pharmaceutical composition comprising the drug. In some embodiments, contacting is administering to the subject.

### Treating a Drug-Resistant Disease in a Subject

By another aspect, there is provided a method of treating a drug-resistant disease in a subject in need thereof, the method comprising administering to the subject a cannabinoid or derivative thereof and the drug, thereby treating the drug-resistant disease in the subject.

By another aspect, there is provided a cannabinoid for use in combination with a drug for treating a drug-resistant disease in a subject in need thereof.

By another aspect, there is provided a drug for use in combination with a cannabinoid for treating a drug-resistant disease in a subject in need thereof.

In some embodiments, the drug-resistant disease is a pathogen. In some embodiments, the pathogen is an infection of the pathogen. In some embodiments, the pathogen is a pathogenic infection. In some embodiments, the pathogen is a bacterium. In some embodiments, the bacterium is an anti-biotic-resistant bacterium. In some embodiments, the antibiotic-resistant bacterium is MRSA. In some embodiments, the pathogen is a fungus. In some embodiments, the drug-resistant disease is cancer. In some embodiments, the drug-resistant disease is a neurological disease. In some embodiments, the neurological disease is selected from brain cancer, epilepsy, schizophrenia, depression and brain infection. In some embodiments, the neurological disease is selected from epilepsy, schizophrenia, depression and brain infection. In some embodiments, the brain infection is HIV. In some embodiments, the drug-resistant disease is a multidrug-resistant disease. In some embodiments, the drug-resistance is efflux-mediated drug-resistance. In some embodiments, the disease effects a target cell. In some embodiments, a cell of the disease is a target cell.

In some embodiments, a cannabinoid is administered. In some embodiments, THCa is administered. In some embodiments, CBD is administered. In some embodiments a cannabinoid selected from THCa and CBD is administered. In some embodiments, a combination of THCa and CBD is administered. In some embodiments, a cannabinoid selected from THCa, CBD and combination thereof is administered. In some embodiments, the cannabinoid or derivative thereof is administered as part of a pharmaceutical composition. In some embodiments, the pharmaceutical composition is a pharmaceutical composition of the invention. In some embodiments, the pharmaceutical composition is non-psychoactive. In some embodiments, the pharmaceutical composition is devoid of THC. In some embodiments, the pharmaceutical composition is devoid of CBD. In some embodiments, devoid is substantially devoid. In some embodiments, the pharmaceutical composition comprises a therapeutically effective amount of the cannabinoid or derivative thereof. In some embodiments, the pharmaceutical composition comprises a pharmaceutically acceptable carrier, excipient or adjuvant.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the chemicals in serum. Liposomes include emulsions, foams, micelles, insoluble monolayers, liquid crystals, phospholipid dispersions, lamellar layers and the like. Liposomes for use with the presently described compositions are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal.

The dosage administered will be dependent upon the age, health, and weight of the recipient, kind of concurrent treatment, if any, frequency of treatment, and the nature of the effect desired.

In some embodiments, the cannabinoid and drug are administered concomitantly. In some embodiments, the cannabinoid is administered prior to the drug. In some embodiments, the cannabinoid is administered at least 0.5 1, 2, 3, 4, 5, 6, 7, 8, 9, 10, 12, 18, 24, 48, 72 or 96 hours before the drug. Each possibility represents a separate embodiment of the invention. In some embodiments, the cannabinoid is administered not more than 0.5 1, 2, 3, 4, 5, 6, 7, 8, 9, 10, 12, 18, 24, 48, 72 or 96 hours before the drug. Each possibility represents a separate embodiment of the invention. In some embodiments, the cannabinoid is administered before the drug at a time sufficient for the cannabinoid to decrease efflux from a target cell of the subject.

In some embodiments, the ratio of cannabinoid to drug is at least 100:1, 50:1, 25:1, 20:1, 15:1, 10:1, 5:1, 4:1, 3:1, 2:1, 1:1, 1:2, 1:3, 1:4, 1:5, 1:10, 1:15, 1:20, 1:25, 1:50, or 1:100. Each possibility represents a separate embodiment of the invention. In some embodiments, the ratio of cannabinoid to drug is at most 100:1, 50:1, 25:1, 20:1, 15:1, 10:1, 5:1, 4:1, 3:1, 2:1, 1:1, 1:2, 1:3, 1:4, 1:5, 1:10, 1:15, 1:20, 1:25, 1:50, or 1:100. Each possibility represents a separate embodiment of the invention.

In some embodiments, the pharmaceutical composition comprises enriched THCa, CBD or both. In some embodiments, THCa is enriched in the pharmaceutical composition. In some embodiments, CBD is enriched in the pharmaceutical composition. In some embodiments, the pharmaceutical composition comprises enriched THCa. In some embodiments, the pharmaceutical composition comprises enriched CBD. In some embodiments, enrichment is at least a 10, 20, 30, 40, 50, 60, 70, 80, 90, 95, 97, 99 or 100% enrichment. Each possibility represents a separate embodiment of the invention. In some embodiments, the dominant cannabinoid in the pharmaceutical composition is THCa. In some embodiments, the dominant cannabinoid in the pharmaceutical composition is CBD. In some embodiments, the cannabinoid is an enriched cannabinoid. In some embodiments, the cannabinoid is a purified cannabinoid. In some embodiments, purified is at least 10, 20, 30, 40, 50, 60, 70, 80, 90, 95, 97, 99 or 100% pure. Each possibility represents a separate embodiment of the invention. In some embodiments, the cannabinoid is an isolated cannabinoid. In some embodiments, the pharmaceutical composition comprises an isolated cannabinoid. In some embodiments, the cannabinoid is isolated from cannabis. In some embodiments, the pharmaceutical composition is substantially devoid of any cannabinoid other than the cannabinoid of the invention.

In some embodiments, the subject is a mammal. In some embodiments, the subject is a veterinary animal. In some embodiments, the subject is a human. In some embodiments, the subject suffers from a drug-resistant disease or condition. In some embodiments, the subject suffers from drug-resistant cancer. In some embodiments, the drug-resistant disease or condition is a multi-drug resistant disease or condition. In some embodiments, the subject suffers from a disease characterized by efflux-mediated resistance. In some embodiments, the subject suffers from a drug resistant infection. In some embodiments, the infection is a bacterial infection. In some embodiments, the bacterial infection is a MRSA infection. In some embodiments, the infection is a fungal infection. In some embodiments, the infection is a viral infection. In some embodiments, the infection is an antibiotic resistant infection.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

### Pharmaceutical Compositions and Kits

By another aspect, there is provided a pharmaceutical composition comprising a pharmaceutically acceptable carrier, excipient or adjuvant, a cannabinoid or derivative thereof, and a drug.

By another aspect, there is provided a pharmaceutical composition comprising a cannabinoid or derivative thereof for use in at least one of:
a. inhibiting efflux;
b. sensitizing a cell to a drug; and
c. treating a drug-resistant condition or disease in a subject by coadministration with the drug.

By another aspect, there is provided a kit comprising a cannabinoid or derivative thereof and at least one of:
a. a drug; and
b. a label stating the cannabinoid or derivative thereof is for use in combination with a drug.

In some embodiments, the pharmaceutical composition comprises THCa. In some embodiments, the pharmaceutical composition comprises CBD. In some embodiments, the pharmaceutical composition is non-psychoactive. In some embodiments, the pharmaceutical composition is substantially devoid of THC. In some embodiments, the pharmaceutical composition is devoid of THC. In some embodiments, the pharmaceutical composition is enriched for the cannabinoid. In some embodiments, the pharmaceutical composition is enriched for THCa. In some embodiments, the pharmaceutical composition is enriched for CBD. In some embodiments, THCa is enriched in the pharmaceutical composition. In some embodiments, the cannabinoid is enriched in the pharmaceutical composition. In some embodiments, CBD is enriched in the pharmaceutical composition.

In some embodiments, the drug is selected form an anti-cancer drug, an antibiotic, an anti-fungal, an antiepileptic, an antipsychotic and an anti-inflammatory. In some embodiments, the drug is selected from an anti-cancer drug, an antibiotic, an anti-fungal and an antipsychotic. In some embodiments, the drug is not an antiepileptic nor an anti-inflammatory drug. In some embodiments, the drug is an anti-cancer drug. In some embodiments, the drug is a chemotherapeutic drug. In some embodiments, the drug is an antibiotic. In some embodiments, the drug is an anti-fungal. In some embodiments, the drug is an antiepileptic drug. In some embodiments, the drug is an antipsychotic drug. In some embodiments, the drug is an anti-inflammatory drug.

In some embodiments, the drug is in a reduced dose in the pharmaceutical composition. In some embodiments, a reduced dose is reduced as compared to the standard dose. In some embodiments, a reduced dose is reduced as compared to a dose administered without the cannabinoid. In some embodiments, the dose is reduced by at least 10, 20, 30, 40, 50, 60, 70, 80, 90, 95, 97, or 99%. Each possibility represents a separate embodiment of the invention.

In some embodiments, the pharmaceutical composition is for use in inhibiting efflux. In some embodiments, pharmaceutical composition is for inhibiting efflux from a cell. In some embodiments, pharmaceutical composition is for inhibiting efflux through a membrane channel. In some embodiments, the pharmaceutical composition is for inhibiting efflux form a cell in a subject.

In some embodiments, pharmaceutical composition is for use in sensitizing a cell to a drug. In some embodiments, the cell is a drug-resistant cell. In some embodiments, the cell is in a subject. In some embodiments, cell is ex vivo. In some embodiments, the pharmaceutical composition is for use in reducing the dose of a drug. In some embodiments, the pharmaceutical composition has a drug-sparing effect. In some embodiments, the pharmaceutical composition is for converting a non-responsive cell or disease to a responsive cell or disease. In some embodiments, the pharmaceutical composition is for making an already responsive cell or disease respond more strongly to the drug. In some embodiments, a stronger response comprises an improvement in at least one symptom as compared to a subject's condition without the pharmaceutical composition.

In some embodiments, pharmaceutical composition is for use in treating a drug resistant disease or condition. In some embodiments, the drug-resistant disease or condition is in a subject. in some embodiments, the treating is of a subject in need thereof. In some embodiments, the pharmaceutical composition is for use in coadministration with the drug. In some embodiments, the pharmaceutical composition is for use in treating a drug-resistant disease or condition by coadministration with the drug. In some embodiments, the coadministration is concomitant. In some embodiments, the coadministration comprises administering the pharmaceutical composition of the invention before the drug.

In some embodiments, the cannabinoid and drug are in a single dose form. As used herein, the term “single dose form” refers to a pharmaceutical product in the form in which it is to be taken. Thus, a single dose form of two agents, is a single dose that can be taken that contains both agents. For example, a single pill, chewable, film, injection, patch or transfusion that contains both the cannabinoid and the drug would be considered a single dose form. In some embodiments, the pharmaceutical composition is in the form of solution, suspension, tablets, chewable tablets, capsules, syrups, films, intranasal sprays, suppositories, transdermal patches, among other types of pharmaceutical compositions. Each possibility represents a separate embodiment of the invention. In some embodiments, the pharmaceutical composition is a long acting, controlled release, extended release or slow release formulation. Each possibility is a separate embodiment of the invention.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

### Synergistic Cytotoxic Effects of Doxorubicin and THCa in Resistant Ovarian Cancer Cells

A non-resistant ovarian cancer cells lines (OVCAR8) and a resistant ovarian cancer cells line (NAR) were exposed to various concentrations of Doxorubicin (Dox), Tetrahydrocannabinolic acid (THCa) and combinations thereof. The cells were exposed to the treatment agents for 4 hours, after which the media was replaced. Cell survival/death levels were measured after 72 hours by XTT. The percent of cell survival was determined as the absorbance signal with subtracted background (media only) and normalized by mock. Each experimental combination was performed in quadruplicate/pentaplicate.

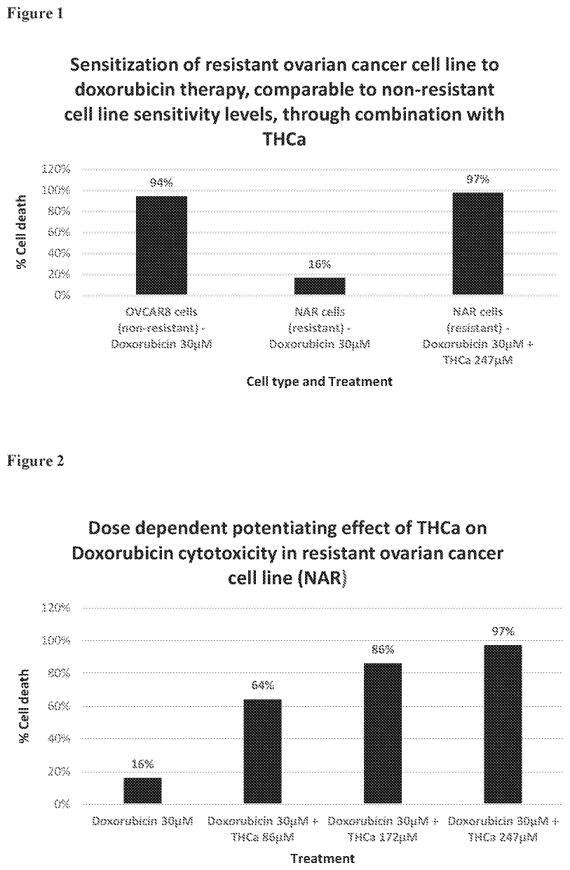

Exposure of non-resistant ovarian cancer cells (OVCAR8) to Doxorubicin (30 μM) resulted in 94% cell death. However, the same exposure to resistant cells (NAR) resulted in only 16% cell death on average. Addition of THCa (247 μM) to the Doxorubicin exposure sensitized the resistant cells and potentiated the lethal efficacy of the Dox treatment, resulting in 97% NAR cell death (FIG. 1).

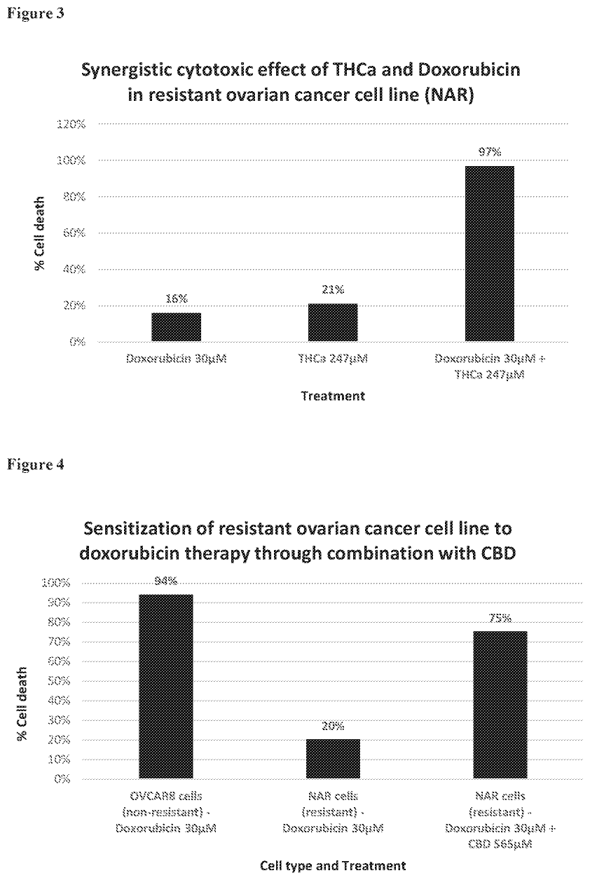

Exposure of ovarian cancer resistant cells (NAR) to Doxorubicin (30 μM) alone resulted in 16% cell death. Addition of increasing concentrations of THCa to the exposure medium resulted in a dose-dependent potentiating effect of THCa on the Doxorubicin cytotoxicity. Specifically, 64% cell death was observed with 86 μM THCa, 86% cell death was observed with 172 μM THCa, and up to 97% cell death was observed with 247 μM THCa (FIG. 2). These results show a clear synergistic effect between the cytotoxic effects of Doxorubicin and THCa on resistant ovarian cancer cells, as the cell death rate in the combination arm far exceeded the additive (accumulated) cell death rates achieved with each agent alone (FIG. 3).

### Example 2

### Potentiating Effect of CBD on Doxorubicin Cytotoxicity in Resistant Ovarian Cancer Cells

A non-resistant ovarian cancer cells line (OVCAR8) and a resistant ovarian cancer cell line (NAR) were exposed to various concentrations of Doxorubicin (Dox), Cannabidiol (CBD) and combinations thereof. The cells were exposed to the treatment agents for 4 hours, after which the media was replaced. Cell survival/death levels were measured after 72 hours by XTT. The percent of cell survival was determined as the absorbance signal with subtracted background (media only) and normalized by mock. Each experimental combination was performed in quadruplicate/pentaplicate.

Exposure of non-resistant ovarian cancer cells (OVCAR8) to Doxorubicin (30 μM) resulted in 94% cell death. However, the same exposure of resistant cells (NAR) resulted in only 20% cell death on average. Addition of CBD (565 μM) to the Doxorubicin exposure sensitized the resistant cells and potentiated the lethal efficacy of the Dox treatment, resulting in 75% NAR cell death (FIG. 4).

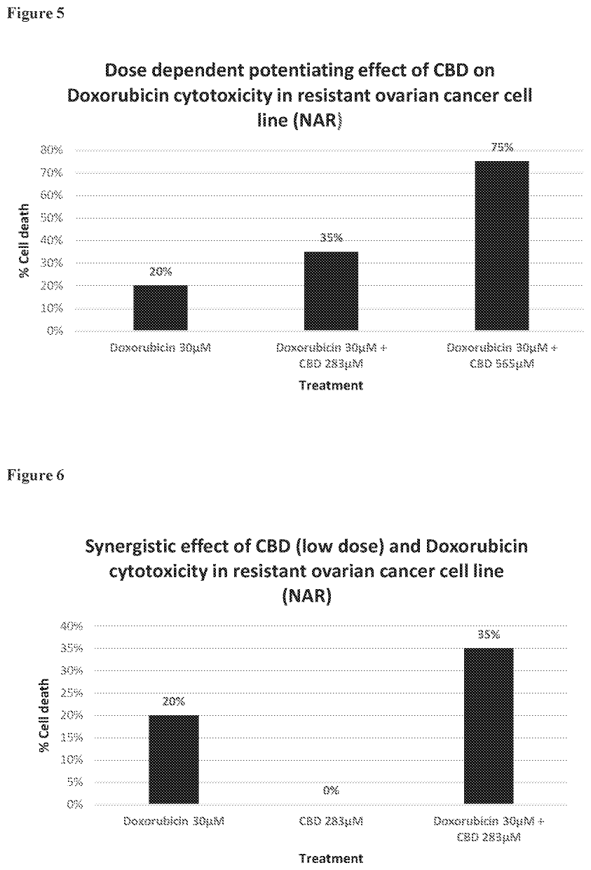

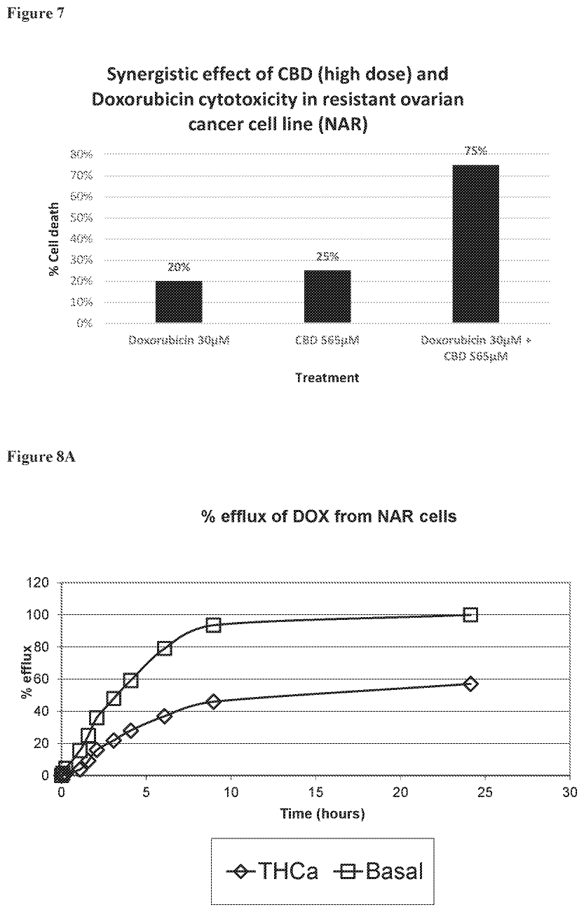

Exposure of ovarian cancer resistant cells (NAR) to Doxorubicin (30 μM) resulted in 20% cell death on average. Addition of increasing concentrations of CBD to the exposure medium resulted in a dose-dependent potentiating effect of CBD on the Doxorubicin cytotoxicity, specifically 35% cell death was observed with 283 μM CBD and 75% cell death with 565 μM CBD (FIG. 5). These results show a clear synergistic effect between the cytotoxic effects of Doxorubicin and CBD on resistant ovarian cancer cells, as the cell death rate in the combination arm far exceeded the additive (accumulated) cell death rates achieved with each agent alone (FIG. 6). The synergistic effect was enhanced when a higher dose of CBD was used (FIG. 7).

Interestingly, when another cannabinoid, Cannabinol (CBN), was tested no effect on Dox toxicity was observed. Dox was once again found to cause 20% cell death on average to resistant NAR cells, but the addition of CBN (674 or 270 μM) did not significantly enhance cell death. This result indicates that the sensitizing effects of THCa and CBD are not shared by all cannabinoids.

### Example 3

### Efflux Pump Inhibition by THCa or CBD in Resistant Ovarian Cancer Cells (NAR)

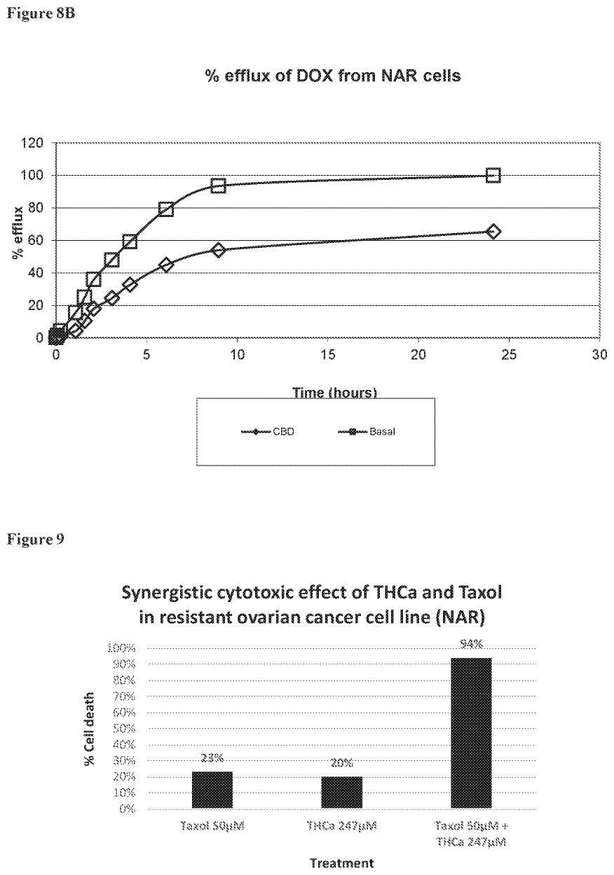

A resistant ovarian cancer cell line (NAR) was pre-treated with sub-lethal concentrations of Doxorubicin (Dox), and then incubated in media with and without THCa or CBD for time periods ranging from 5 minutes to 24 hours (a total of 12 time points were measured). The ability of the cells to efflux Dox into the media was measured and compared between the cells grown in the presence and absence of THCa (FIG. 8A) or CBD (FIG. 8B). FIG. 8A shows the significant inhibitory effect THCa has on the functionality of NAR cell's efflux pumps, as efflux levels decreased by nearly 50%. FIG. 8B shows the significant inhibitory effect CBD has on the functionality of NAR cell's efflux pumps, as efflux levels decreased by nearly 40%.

### Example 4

### Potentiating Effect of THCa on Taxol Cytotoxicity in Resistant Ovarian Cancer Cells

These experiments were repeated with a second chemotherapeutic drug, taxol, and similar results were observed.

The resistant ovarian cancer cells line (NAR) was exposed to various concentrations of Taxol (Paclitaxel), Tetrahydrocannabinolic acid (THCa) and combinations thereof. The cells were exposed to the treatment agents for 4 hours, after which the media was replaced. Cell survival/death levels were measured after 72 hours by XTT. The percent of cell survival was determined as the absorbance signal with subtracted background (media only) and normalized by mock. Each experimental combination was performed in quadruplicate/pentaplicate.

Exposure of ovarian cancer resistant cells (NAR) to Taxol (50 μM) alone resulted in 23% cell death. Exposure to THCa (247 μM) alone resulted in 20% cell death. Addition of THCa to the exposure medium resulted in a dose-dependent potentiating effect of THCa on the Taxol cytotoxicity. Specifically, 94% cell death was observed with 247 μM THCa and 50 μM Taxol (FIG. 9). These results show a clear synergistic effect between the cytotoxic effects of Taxol and THCa on resistant ovarian cancer cells.

### Example 5

### Potentiating Effect of THCa on Cisplatin Cytotoxicity in Resistant Ovarian Cancer Cells

These experiments were repeated with a third chemotherapeutic drug, Cisplatin, and similar results were observed.

The resistant ovarian cancer cells line (NAR) was exposed to various concentrations of Cisplatin, Tetrahydrocannabinolic acid (THCa) and combinations thereof. The cells were exposed to the treatment agents for 4 hours, after which the media was replaced. Cell survival/death levels were measured after 72 hours by XTT. The percent of cell survival was determined as the absorbance signal with subtracted background (media only) and normalized by mock. Each experimental combination was performed in quadruplicate/pentaplicate.

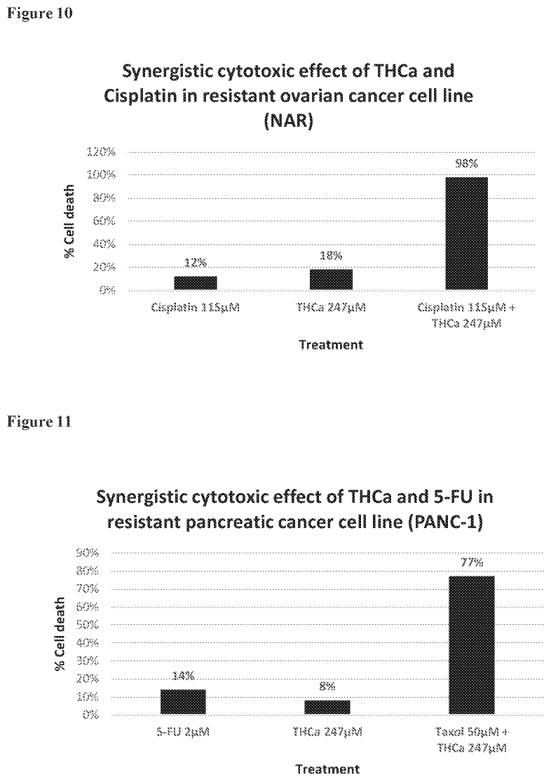

Exposure of ovarian cancer resistant cells (NAR) to Cisplatin (115 μM) alone resulted in 12% cell death. Exposure to THCa (247 μM) alone resulted in 18% cell death. Addition of THCa to the exposure medium resulted in a dose-dependent potentiating effect of THCa on the Cisplatin cytotoxicity. Specifically, 94% cell death was observed with 247 μM THCa and 115 μM Cisplatin (FIG. 10). These results show a clear synergistic effect between the cytotoxic effects of Cisplatin and THCa on resistant ovarian cancer cells.

### Example 6

### Synergistic Cytotoxic Effects of 5-FU and THCa in Resistant Pancreatic Cancer Cells

A resistant pancreatic cancer cells line (PANC-1) was exposed to fluorouracil (5-FU), Tetrahydrocannabinolic acid (THCa) and combination thereof. The cells were exposed to the treatment agents for 4 hours, after which the media was replaced. Cell survival/death levels were measured after 72 hours by XTT. The percent of cell survival was determined as the absorbance signal with subtracted background (media only) and normalized by mock. Each experimental combination was performed in quadruplicate/pentaplicate.

Exposure of pancreatic cancer resistant cells (PANC-1) to 5-FU (2 μM) alone resulted in 14% cell death. Exposure of pancreatic cancer resistant cells (PANC-1) to THCa (247 μM) alone resulted in 8% cell death. Addition of THCa to the exposure medium resulted in a dose-dependent potentiating effect of THCa on the 5-FU cytotoxicity. Specifically, 77% cell death was observed with 247 μM THCa and 2 μM 5-FU (FIG. 11). These results show a clear synergistic effect between the cytotoxic effects of 5-FU and THCa on resistant pancreatic cancer cells, as the cell death rate in the combination arm far exceeded the additive (accumulated) cell death rates achieved with each agent alone.

Resistant pancreatic cancer cells are tested for synergistic effect between 5-FU and CBD as well.

### Example 7

### Synergistic Cytotoxic Effects of Vincristine and THCa in Resistant Lung Cancer Cells

A multi-drug resistant small cell lung cancer cells line (NCI-H69/LX4) was exposed to Vincristine, Tetrahydrocannabinolic acid (THCa) and combination thereof. The cells were exposed to the treatment agents for 4 hours, after which the media was replaced. Cell survival/death levels were measured after 72 hours by XTT. The percent of cell survival was determined as the absorbance signal with subtracted background (media only) and normalized by mock. Each experimental combination was performed in quadruplicate/pentaplicate.

Exposure of lung cancer resistant cells (NCI-H69/LX4) to Vincristine (10 μM) alone resulted in 17% cell death. Exposure of lung cancer resistant cells (NCI-H69/LX4) to THCa (247 μM) alone resulted in 12% cell death. Addition of THCa to the exposure medium resulted in a dose-dependent potentiating effect of THCa on the Vincristine cytotoxicity. Specifically, 95% cell death was observed with 247 μM THCa and 10 μM Vincristine (FIG. 12). These results show a clear synergistic effect between the cytotoxic effects of Vincristine and THCa on resistant lung cancer cells, as the cell death rate in the combination arm far exceeded the additive (accumulated) cell death rates achieved with each agent alone.

Resistant lung cancer cells are tested for synergistic effect between 5-FU and CBD as well.

### Example 8

### THCa Increases MRSA Sensitivity to Multiple Antibiotic Treatments in a Synergistic Manner

Methicillin-resistant Staphylococcus aureus (MRSA) is a major cause of serious nosocomial infections that show resistance to multiple types of antibiotic treatments. The ability of Tetrahydrocannabinolic acid (THCa) to increase the antibiotic sensitivity of pathogenic MRSA cells to various antibiotics was investigated. THCa, and tetracycline, gentamycin, chloramphenicol, ciprofloxacin, rifampicin, and vancomycin were tested alone and in combination for their ability to eradicate resistant MRSA pathogens.

A single S. aureus ATCC33591 colony was grown in 4 ml of Mueller-Hinton (MH) broth (Oxoid) at 225 r.p.m. at 37° C. overnight, and the resulting overnight culture was diluted 1:100 in MH and incubated at 37° C. at 225 r.p.m. until an OD600 of 0.6 was reached. The bacterial cells were collected by centrifugation, washed once with saline (0.9% NaCl) and resuspended in saline solution. To study the effect of THCa on the sensitivity of MRSA to the antibiotics, MRSA were incubated with tetracycline, gentamycin, chloramphenicol, ciprofloxacin, rifampicin, and vancomycin with and without the presence of THCa, and with THCa alone (without antibiotic treatment) for 4 hours. The relevant antibiotics concentration for each type of antibiotic treatment was determined according to previously shown MIC (Minimal Inhibitory Concentration) values for the corresponding antibiotics and the used strain of MRSA. Post incubation, samples were serially diluted with 0.9% NaCl, and 10 μl of each dilution was spotted onto MIR agar plates and incubated overnight at 37° C. and the numbers of surviving bacteria were determined by counting the resulting CFUs.

6% cell death of MRSA cells was observed following incubation with THCa (88 μM) alone. Table 1 provides the lethal effect on MRSA cells of each antibiotic alone (at MIC values), and in the presence of 88 μM THCa. None of the antibiotics alone produced even 20% cell killing. By contrast, the weakest synergistic effect was observed for THCa with tetracycline which still caused 59% MRSA cell death. The most potent combination, THCa with rifampicin, produced 92% MRSA cell death. The results show a clear synergistic effect between the lethal effects of the selected antibiotics on MRSA cells and the lethal effects of THCa on these cells, as the cell death rate in the combination arm far exceeded the additive (accumulated) cell death rates achieved with each agent alone.

TABLE 1
Percentage death of MRSA cells following incubation with indicated
antibiotics alone, THCa alone and the combination of antibiotics + THCa.

| | Antibiotics | THCa (88 μM) | Antibiotics + |
|---|---|---|---|
| Type of Antibiotics | alone | alone | THCa (88 μM) |
| Rifampicin (0.4 mg/liter) | 17% | 6% | 92% |
| Vancomycin (5 mg/liter) | 8% | 6% | 78% |
| Ciprofloxacin (5 mg/liter) | 14% | 6% | 88% |
| Gentamycin (20 mg/liter) | 15% | 6% | 85% |
| Tetracycline (50 mg/liter) | 7% | 6% | 59% |
| Chloramphenicol (50 | 12% | 6% | 67% |
| mg/liter) | | | |

### Example 9

### CBD Increases MRSA Sensitivity to Multiple Antibiotic Treatments in a Synergistic Manner

The ability of cannabidiol (CBD) to increase the antibiotic sensitivity of pathogenic MRSA cells to various antibiotics was investigated. CBD and tetracycline, gentamycin, chloramphenicol, ciprofloxacin, rifampicin, and vancomycin were tested alone and in combination for their ability to eradicate resistant MRSA pathogens.

A single S. aureus ATCC33591 colony was grown in 4 ml of Mueller-Hinton (MH) broth (Oxoid) at 225 r.p.m. at 37° C. overnight, and the resulting overnight culture was diluted 1:100 in MH and incubated at 37° C. at 225 r.p.m. until an OD600 of 0.6 was reached. The bacterial cells were collected by centrifugation, washed once with saline (0.9% NaCl) and resuspended in saline solution. To study the effect of CBD on the sensitivity of MRSA to the antibiotics, MRSA were incubated with tetracycline, gentamycin, chloramphenicol, ciprofloxacin, rifampicin, and vancomycin with and without the presence of CBD, and with CBD alone (without antibiotic treatment) for 4 hours. The relevant antibiotics concentration for each type of antibiotic treatment was determined according to previously shown MIC values for the corresponding antibiotics and the used strain of MRSA. Post incubation, samples were serially diluted with 0.9% NaCl, and 10 μl of each dilution was spotted onto MH agar plates and incubated overnight at 37° C. and the numbers of surviving bacteria were determined by counting the resulting CFUs.

9% cell death of MRSA cells was observed following incubation with CBD (565 μM) alone. Table 2 provides the lethal effect on MRSA cells of each antibiotic alone (at MIC values), and in the presence of 565 μM CBD. None of the antibiotics alone produced even 20% cell killing. By contrast, the weakest synergistic effect was observed for CBD with tetracycline which still caused 42% MRSA cell death. The most potent combination, CBD with rifampicin, produced 73% MRSA cell death. The results show a clear synergistic effect between the lethal effects of the selected antibiotics on MRSA cells and the lethal effects of CBD on these cells, as the cell death rate in the combination arm far exceeded the additive (accumulated) cell death rates achieved with each agent alone.

TABLE 2
Percentage death of MRSA cells following incubation with indicated
antibiotics alone, CBD alone and the combination of antibiotics + CBD.

| | Antibiotics | CBD (565 μM) | Antibiotics + |
|---|---|---|---|
| Type of Antibiotics | alone | alone | CBD (565 μM) |
| Rifampicin (0.4 mg/liter) | 17% | 9% | 73% |
| Vancomycin (5 mg/liter) | 8% | 9% | 61% |
| Ciprofloxacin (5 mg/liter) | 14% | 9% | 54% |
| Gentamycin (20 mg/liter) | 15% | 9% | 58% |
| Tetracycline (50 mg/liter) | 7% | 9% | 42% |
| Chloramphenicol (50 | 12% | 9% | 58% |
| mg/liter) | | | |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-9. (canceled)

10. A method of inhibiting efflux from a cell through a membrane channel, comprising contacting said cell with tetrahydrocannabinolic acid (THCa), thereby inhibiting efflux from a cell.

11. The method of claim 10, further comprising contacting said cell with cannabidiol (CBD).

12. The method of claim 10, wherein said cell is a chemotherapy resistant cell.

13. The method of claim 12, wherein said chemotherapy resistant cell is a multi-drug-resistant (MDR) cell.

14. The method of claim 12, wherein said cell is a cancer cell, and wherein said cancer is selected from ovarian cancer, pancreatic cancer, and lung cancer.

15. The method of claim 10, wherein said cell is an antibiotic resistant cell.

16. The method of claim 15, wherein said antibiotic resistant cell is a Methicillin-resistant Staphylococcus aureus (MRSA) cell.

17. The method of claim 10, wherein said cell is a drug-resistant cell and said method is a method of sensitizing a drug-resistant cell to said drug.

18. (canceled)

19. (canceled)

20. The method of claim 17, wherein said drug-resistant cell comprises efflux-mediated drug-resistance.

21. The method of claim 17, wherein said drug is selected from an anti-cancer drug, an antibiotic, an antipsychotic, an anti-androgen, an immunosuppressant, a lipid lowering drug, an antihistamine, a steroid, a dopamine antagonist, a protein inhibitor, a cardiac drug, an antiemetic, an antidiarrheal, and antigout and an anti-fungal.

22. The method of claim 21, wherein said anti-cancer drug is selected from a chemotherapeutic, an anthracycline, a vinca alkaloid, a taxane, a podophyllotoxin derivative, a PARP inhibitor, a folate based anti-metabolite, an alkylating agent, an epothilone, a histone deacetylase inhibitor, a topoisomerase I or II inhibitor, a kinase inhibitor, a nucleotide analog or precursor analog, a podophyllotoxin derivative, a platinum based agent, or a retinoid.

23. The method of claim 22, wherein said anticancer drug is a chemotherapeutic selected from doxorubicin, paclitaxel, cisplatin and 5-FU.

24. The method of claim 21, wherein said antibiotic drug is selected from tetracycline, gentamycin, chloramphenicol, ciprofloxacin, rifampicin, and vancomycin.

25. The method of claim 17, wherein said drug-resistant cell is a cancer cell in a subject, and the method further comprises administering said drug to said subject, thereby treating cancer in said subject.

26. The method of claim 25, wherein said drug-resistant cancer is a multidrug-resistant cancer.

27. The method of claim 25, wherein said cancer is selected from ovarian cancer, pancreatic cancer, and lung cancer.

28. The method of claim 17, wherein said drug-resistant cell is a pathogen in a subject, and the method further comprises administering said drug to said subject, thereby treating said an infection by said pathogen in said subject.

29. The method of claim 28, wherein said pathogen is an antibiotic resistant bacterium.

30. The method of claim 29, wherein said antibiotic resistant bacterium is MRSA.

31. The method of claim 25, wherein said administering is concomitant with said contacting or subsequent to said contacting.

32-36. (canceled)
